(19) United States
(12) Patent Application Publication
(10) Pub. No.: US 2018/0221072 A1
(43) Pub. Date: Aug. 9, 2018

(54) Local delivery device for targeted therapeutic effect

(71) Applicants: KANNAN P, Sivaganga (IN); SWARNA S, Sivaganga (IN)

(72) Inventors: KANNAN P, Sivaganga (IN); SWARNA S, Sivaganga (IN)

(21) Appl. No.: 15/505,939

(22) PCT Filed: Aug. 25, 2015
(86) PCT No.: PCT/IB2015/056424

§ 371 Date: Feb. 23, 2017

(30) Foreign Application Priority Data
Aug. 25, 2014 (IN) 4138/CHE/2014

(51) Int. Cl. A61B 17/86; A61B 17/88

(52) U.S. Cl. CPC A61B 17/864; A61B 17/8625; A61B 2017/00004; A61B 17/8875; A61B 17/862; A61B 17/8615

(57) ABSTRACT

An embodiment herein provides a local delivery device for targeted therapeutic effect. Accordingly, the local delivery device comprises a cap preferably a hemispherical cap, a locking part and a shaft part, wherein the local delivery device can be of a centre tightened local delivery device or a periphery tightened delivery device, wherein the hemispherical cap comprises a screw driver slot at its centre or at its periphery. The locking part has its outer surface threaded and includes a canister to accommodate the desired agent for delivery or can be made of substance intended with or without additives and bases. The locking part may be attached to the hemispherical cap. The shaft part includes unthreaded or threaded configuration and attached to the locking part. A locking hole provides an aperture to introduce the local delivery device to the plate or other implants of choice and bone underneath.

### FIELD OF INVENTION

The embodiments herein generally relate to medical devices for therapuetic effect, more specifically, it relates to a local delivery device for targeted therapeutic effect, particularly pertains to an implant device to deliver physical, pharmacological, biological active substances, piezo electric materials, ion diffusion, galvanic particulates, nano particles or things to aid in fracture healing, clearing infection or malignancy and to strengthen bone and soft tissues.

### BACKGROUND OF THE INVENTION

Bones are rigid organs that constitute a part of the musculo-skeletal system. They support and protect various organs of the body, produce red and white blood corpuscles and store minerals. Bones are consistently under many kinds of forces naturally applied on them, but when the forces are too strong the bones may break.

A bone plate is a metal plate, which is used to reconstruct a bone that has been fractured. The metal plates include perforations for the insertion of screws and are also used to immobilize fracture segments. The usage of bone plates has revolutionized the trend towards the surgical approach of bone fractures.

Hence, open reduction and internal fixation and minimally invasive techniques are used to treat muscular skeleton problems like fracture of bones, deformity correction, articular reconstruction, ligament repair, treating tumors, fusing joints and to treat periprosthetic fractures, geriatric and paediatric applications.

Usage of plate osteosynthesis either alone or with other modalities of treatment for the above mentioned conditions in an established practice, with an advent of locking and locking compression plates has evolved much. Still complications like non-union, delayed union of fractures, infection, periprosthetic fractures, osteoporosis, tumor recurrence, and soft tissue damages are prevalent in significant percentage.

But in order to make injured bones healthier, therapeutic agents are essential. Bone implants with or without screw can include a slot for the delivery of medicine to the healing portion. The medicines can be delivered in to the desired portion through a needle from a syringe or using some other delivery mechanisms the medicines are attempted to sent in to the wounded portion.

Many of these devices have a formidable challenge of local delivery of pharmacological, biological factors or bioactive ingredients and other physical methods at the site of requirement.

In order to overcome the problem associated with the prior art, research has been focused towards developing a delivery device for a desired therapeutic effect. But, the problems associated with the prior arts are that the antibiotic impregnated bone cement beads implantation for example has the short coming of restricted choice of antibiotics compatible with the cement, restricted dosage of drug, unpredictable elution kinetics, antibiotic resistances developed by the micro-organisms to the drug implanted and may need at least two surgery with soft tissue dissection for implantation and exit as in case of PMMA cement, more over it may also serve as a nidus for infection defeating the very purpose. All these produce more trauma to the patient surgically and psychologically. Moreover, localizing the targeted delivery device with or without the bone locking plate is not present to deliver therapeutic agents as a promising delivery device and tightening mechanisms for such device are not providing proper delivery of therapeutic agents in the existing art regarding the aspects of consistent localization, ease of application and removal during various stages of healing, delivery of multiple effects including physical methods such as piezo electrical etc., and its accommodation with the principles of internal fixation.

Therefore there exist a need in the art to develop a local delivery device for targeted therapeutic effect with multi delivery system, to overcome the problem associated with the prior art, and also to provide an exclusive delivery device with a tightening mechanism to deliver physical, pharmacological, biological active substances, piezo electric materials, ion diffusion, galvanic particulates, nano particles or things to aid in fracture healing, clearing or malignancy and to strengthen bone and soft tissues.

### OBJECTS OF THE INVENTION

Some of the objects of the present disclosure are described herein below:

A main object of the present invention is to provide a local delivery device, wherein the delivery device is made of metal, alloy, polymer, bio-absorbable material, material of therapeutic agent itself with or without necessary bases, additives and adjuvant or combination of them.

Another object of the present invention is to provide a local delivery device with multi delivery system wherein parts of the local delivery device such as a hemispherical cap, a locking part and a shaft part can be used in a combination or together in a bone locking plate, intramedullary nail or in other implants of choice and also the parts of the delivery device can be used in combination or altogether as an individual delivery device.

Still another object of the present invention is to provide a centre tightened local delivery device or a periphery tightened local delivery device.

Yet another object of the present invention is to provide a locking part with its outer surface is threaded and made of metal, alloy, polymers, bio-absorbable materials or the substance of intend itself with or without additives and bases. Further, the locking part can be used individually with the local delivery device or in the bone locking plate or other implants of choice.

Another object of the present invention is to provide a shaft to be attached to the locking part and the shaft part is with or without threaded portion.

Another object of the present invention is to provide a hemispherical cap and a locking part as a local delivery device for targeted therapeutic effect; or a locking part and a shaft part as a local delivery device for targeted therapeutic effect; or a hemispherical cap, a locking part and a shaft part, all together as a local delivery device for targeted therapeutic effect.

The other objects and advantages of the present invention will be apparent from the following description when read in conjunction with the accompanying drawings which are incorporated for illustration of preferred embodiments of the present invention and are not intended to limit the scope thereof.

### SUMMARY OF THE INVENTION

In view of the foregoing, an embodiment herein discloses a local delivery device for targeted therapeutic effect. In an embodiment, a local delivery device is made of metal, alloy, polymer, bio-absorbable material, material of therapeutic agent itself with or without necessary bases, additives and adjurants or combination of them. Accordingly, the local delivery device comprises a cap preferably a hemispherical cap, a locking part and a shaft part, wherein the hemispherical cap comprises a screw driver slot at its centre or at its periphery, wherein a locking hole provides an aperture to introduce the local delivery device to a plate or other implants of choice and bone underneath. The locking part has its outer surface threaded and includes a canister to accommodate a desired agent for delivery or can be made of an intended with or without additives and bases. The locking part may be attached to the hemispherical cap. The shaft part attached to the locking part includes an unthreaded or a threaded configuration. Accordingly, the local delivery device can be used in a combination or together in a bone locking plate, or other implants of choice or parts of the delivery device can be used in combination or together as an individual delivery device.

According to an embodiment, the local delivery device can be a centre tightened local delivery device or a periphery tightened local delivery device. In the centre tightened local delivery device, a hemispherical cap includes a screw driver slot which can be given in the middle of the hemispherical cap extending optionally down in to the locking part and to the shaft and may extend up to the farthest end. In the periphery tightened local delivery device, the device is tightened at its periphery. The hemispherical cap may have its diameter optionally exceeding or within the breadth of the implant and may have additional apertures for dispersal of the therapeutic agent.

In an embodiment, the locking part can either be alone or in combination with the hemispherical cap with a screw driver slot insertion, locks only with the implant especially plates thereby sparing the bone underneath. The hemispherical cap can be added to the locking part to provide an extra space and can be attached to the locking part in an obtuse manner to reduce stress raiser effect.

In an embodiment, the length of the shaft part is varied depending on the site of an intended delivery and can be inserted in unicortical or bicortical fashion. The shaft part is attached to a locking part and is continuous to make a locked stable structure. The selection of a threaded or an unthreaded configuration of the shaft part can be based on the need.

In an embodiment, the local delivery device can be a solid medicine without any apertures or openings as in case for example piezoelectric substances or may have openings at one or either ends to facilitate delivery of desired effect and can also have an additional aperture openings for intramedullary delivery or an augmented delivery of therapeutic effects.

In an embodiment, the local delivery device for targeted therapeutic effect may be a solid whole body, which are made of an intended substance or their combinations and may also serve as a canister to store and deliver substances of choice. Further, the local delivery device can vary in size and shape according to implant's locking dimensions, in an example may be conical frustum and has matching threads as locking mechanism with implant like bone locking plates or can be otherwise in accordance with the implant's locking dimensions. The mechanism of locking can be adapted to that of the implant or to the bone into which applied and can be a mechanism other than locking threads.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The detailed description is set forth with reference to the accompanying figures. In the figures the use of the same reference numbers in different figures indicates similar or identical items.

FIG. 1a illustrates a perspective view of a centre tightened local delivery device for targeted therapeutic effect, according to an embodiment herein;

FIG. 1b illustrates a perspective view of a periphery tightened local delivery device for targeted therapeutic effect, according to an embodiment herein;

FIG. 2a illustrates a cross sectional view of a centre tightened local delivery device for targeted therapeutic effect, according to an embodiment herein;

FIG. 2b illustrates a cross sectional view of a periphery tightened local delivery device for targeted therapeutic effect, according to an embodiment herein;

FIG. 3a illustrates parts of a centre tightened local delivery device for targeted therapeutic effect, according to an embodiment herein;

FIG. 3b illustrates parts of a periphery tightened local delivery device for targeted therapeutic effect, according to an embodiment herein;

FIG. 4a illustrates a centre tightened local delivery device with a bone locking plate, according to an embodiment herein; and

FIG. 4b illustrates a periphery tightened local delivery device with a bone locking plate, according to an embodiment herein;

### DETAILED DESCRIPTION OF THE PREFERRED EMBODIMENTS

The embodiments herein and the various features and advantageous details thereof are explained more fully with reference to the non-limiting embodiments and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

According to an embodiment, a device for local delivery of targeted therapeutic effect, comprising a cap includes a slot for tightening the device, a locking part includes an optionally threaded outer surface to lock the device with an implant or a treatment part independently. The locking part may also include a mechanism other than threaded outer surface to lock the device with in implant or a trademark part. The locking part along with cap and shaft parts either in combination or alone also includes a canister to accommodate a desired agent for delivery and are also be made of an intended substance with or without additives and bases as a solid one, and a shaft part having a predetermined length extending towards desired location in and around of treatment part. Wherein the slot is provided in center of the cap or in periphery of the cap, wherein the center slot is optionally augumented at its base extending in middle up to the farthest part of the device to deliver the torque effectively, wherein the periphery slot is optionally augumented at its periphery to the fartherst possible to deliver the torque effectively. In an embodiment, a torque controlled driver is used for fastening the device using the slot to prevent complications in the removal of implantation.

In an embodiment, the device is made from a material selected from the group comprising of metal, alloy, polymer, bio-absorbable material, material of therapeutic agent itself with or without necessary bases, additives or adjuvant, or combination of all.

In an embodiment, the cap, locking part and shaft part are joined as non-seperable single unit, wherein the joining is made in a smooth fashion aligned at predetermined angle preferably at obtuse angle to prevent stress raiser effect.

In an embodiment, the cap, locking part and shaft part are provided seperately to use in different combination such as locking part alone or locking part with cap or locking part with shaft part for therapeutic effect.

In an embodiment, the size and shape of said cap, locking part and shaft part are variable based on the application, wherein shape of said cap is either spherical or polygon The cap of the device can be suitably modified in shape and size to suite the needs such as a scaffold for cartilage and tissue regeneration and to accept sutures, cables and S.S wires. Further, the said device can be used as a gap filler in the bone.

In an embodiment, the shaft part includes either threaded outer sufrace or smooth unthreaded outer surface to suite the need of treatment part. In another embodiment, the shaft part includes reverse cutting threads to facilitate the easy removal of the device.

In an embodiment, the cap, locking part and shaft part are provided in solid base made of substance of intend or with space inside to function as a reservior or vehicle for the substance of intend, wherein the shaft, cap and the locking parts optionaly includes one or more apertures to facilitate release of substance, wherein the release of substance from the apertures is regulated by physical structures includes of sieves, membranes or plugs, or by physical and chemical properties of the materials includes of viscosity, charged ions, pH and biological interaction with body fluids, wherein the substance is prefilled in the device or constituted intra-operatively and applied or can replenish or replace the one existing inside the device.

In an embodiment, the device is present in a combination or together in a bone locking plate, or other implants of choice or parts of the delivery device are used in combination or together as an individual delivery device.

Accordingly, the device can be used to deliver physical, pharmacological, biological active substances, piezo electric materials, ion diffusion, galvanic particulates, nano particles or things to aid in fracture healing, clearing infection or malignancy and to strengthen bone and soft tissues, cartilage regeneration, oseoconductive, osteoinductive materials bone graft and bone graft substitutes.

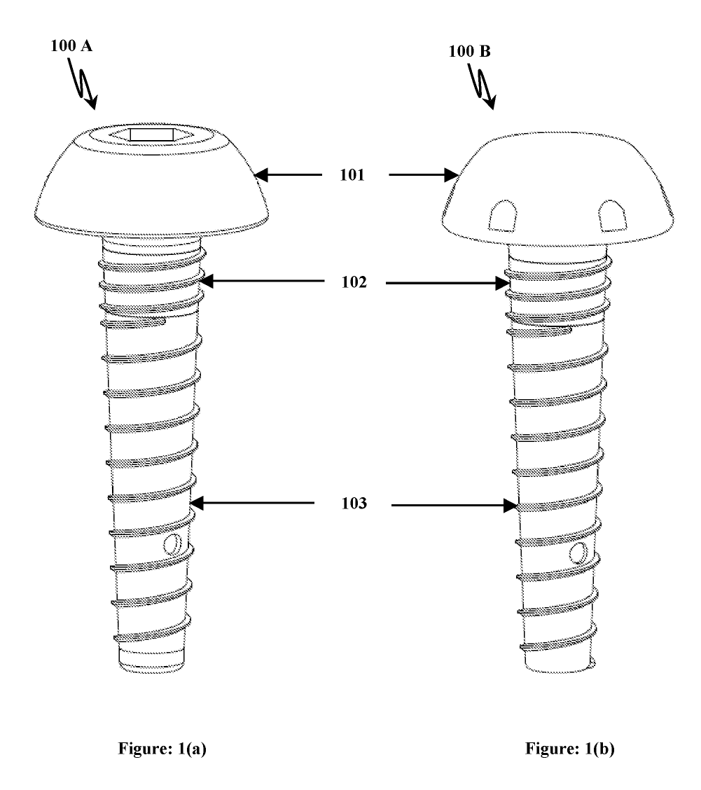

As mentioned, there remains a need for developing a local delivery device for targeted therapeutic effect. The embodiments herein achieve this by providing a local delivery device, wherein parts of the local delivery device can be used in a combination or together along with a bone locking plate and in other implants of choice or can be used in combination or together as an individual delivery device. Referring now to drawings, and more particularly to FIGS. 1a through 4b, where similar reference characters denote corresponding features consistently throughout the figures, there are shown preferred embodiments.

According to an embodiment, FIG. 1a illustrates a perspective view of a centre tightened local delivery device 100A for targeted therapeutic effect. In an embodiment, a perspective view of a periphery tightened local delivery device 100B for targeted therapeutic effect is illustrated in FIG. 1b. According to an embodiment, a centre tightened local delivery device 100A and a periphery tightened local delivery device 100B comprises a cap preferably a hemispherical cap 101, a locking part 102 and a shaft part 103, wherein the local delivery device is made of metal, alloy, polymer, bio-absorbable material, material of therapeutic agent itself with or without necessary bases, additives and adjuvant or combination of them.

In an embodiment, a local delivery device for targeted therapeutic effect can be of a centre tightened local delivery device 100A or a periphery tightened local delivery device 100B.

In an embodiment, the centre tightened local delivery device 100A includes tightening mechanism at a centre of the local delivery device. In an embodiment, the periphery tightened local delivery device 100B includes tightening mechanism at its periphery of the local delivery device.

In an embodiment, the local delivery device can be suitably modified with intend of an independent device to bone without implant for local delivery of desired effect.

In some embodiments, the local delivery device can be a solid medicine without any apertures or openings as in case for example piezoelectric substances or may have openings at one or either ends to facilitate delivery of desired effect and can also have additional apertures openings for an intramedullary delivery or an augmented delivery of the therapeutic effects.

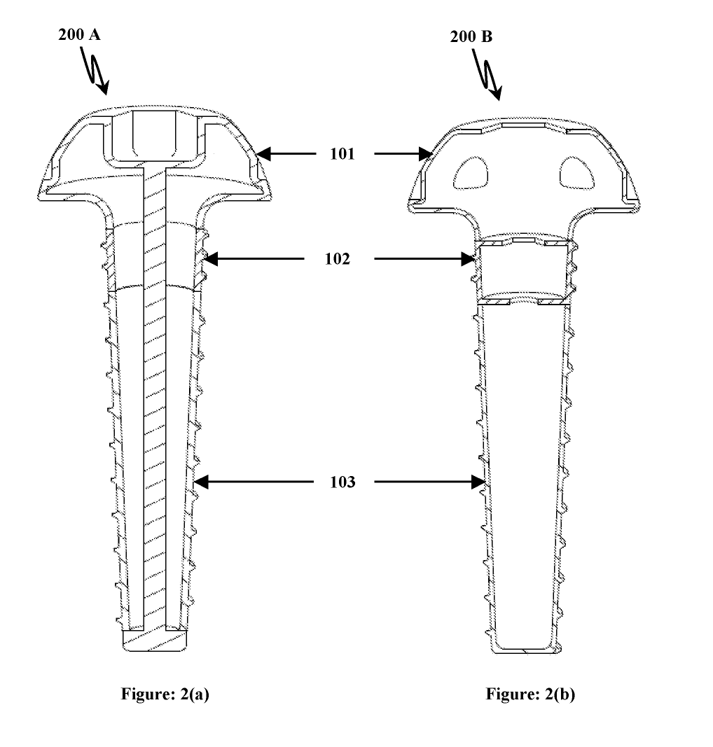

According to an embodiment, FIG. 2a illustrates a cross sectional view 200A of a centre tightened local delivery device for targeted therapeutic effect. In an embodiment, a cross sectional view 200B of a periphery tightened local delivery device for targeted therapeutic effect is illustrated in FIG. 2b.

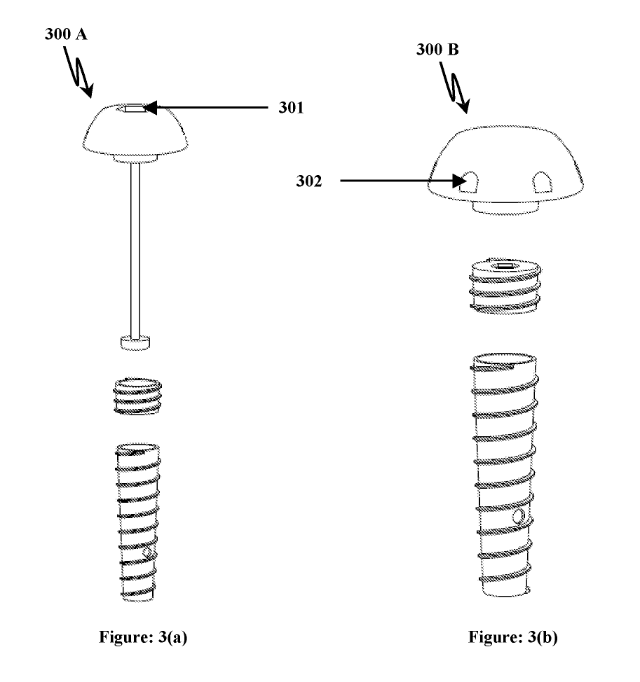

FIG. 3a illustrates parts of a centre tightened local delivery device 300A for targeted therapeutic effect, according to an embodiment. In an embodiment, parts of a periphery tightened local delivery device 300B for therapeutic treatment is illustrated in FIG. 3b. Both the local delivery device includes a hemispherical cap 101, a locking part 102 and a shaft part 103. The hemispherical cap 101 can be added to the locking part 102 to provide an extra space in an obtuse manner to reduce stress raiser effect. The hemispherical cap 101 of the centre tightened local delivery includes a screw driver slot 301 to achieve tightening mechanism at a centre of the hemispherical cap 101. The hemispherical cap 101 of the periphery tightened local delivery device includes a periphery slot 302 to tighten the device at its periphery.

In an embodiment, the screw driver slot 301 can be given in the middle or at its sides of the hemispherical cap in the centre tightening local delivery device 100A and extending optionally down in to the locking part 102 and to the shaft 103 and may extend up to the farthest end. In the periphery tightened local delivery device 100B, the periphery slot 302 can be provided at the periphery of the hemispherical cap 101 to introduce the delivery device to the bone locking plate and bone underneath.

In an embodiment, the centre tightening local delivery device 100A is applied by an above downward fashion using a screw driver through a slot 301 on its top. Hence, the local delivery device can be easy to perform by a surgeon who is routinely familiar with the procedure of screw insertion and torque control of screw insertion.

In an embodiment, the volume of the local delivery device may be increased by giving a smooth contoured convexity in the hemispherical cap 101 on top of the locking part 102. The hemispherical cap 101 may have its diameter optionally exceeding or within the breadth of the implant and may have additional apertures for dispersal of the therapeutic agent.

In an embodiment, the centre tightened local delivery device 100A includes a screwdriver slot 301 which is provided at the top with shape of choice and torque control can be used to protect the centre tightened local delivery device 100A.

In an embodiment, the locking part 102 has its outer surface is threaded and the locking part 102 can be a canister to accommodate the desired agent for delivery or can be made of the substance of intend with or without additives and bases.

According to an embodiment, the locking part 102 can either be alone or in combination with the hemispherical cap 101. The locking part 102 may lock only with the implant especially plates thereby sparing the bone underneath. Hence, the delivery device can be applied in the desired location on the implant even prior to its insertion thus shortening and simplifying operating time and techniques respectively, with proper planning of the delivery of the desired therapeutic effect can be precisely located over the region of necessity without additional dissection and soft tissue injury as it can be inserted by biological techniques of internal fixation. The local delivery device can be added intra operatively after the implantation, or also can be inserted isolatedly in the post operation period based on the need to address complications such as infection, non-union tumor recurrence, osteoporosis etc.,

In an embodiment, the local delivery device is incarcerated to the plate or implant alone through locking mechanism, which may not need a separate surgery for removal and may lessen the soft tissue injury as delivery device does not require any separate procedure as it comes out along with the plate or implant exit.

In an embodiment, the shaft part 103 can be cannulated or solid based on the need. The length of the shaft part 103 is varied depending on the site of the intended delivery and can be inserted in unicortical or bicortical fashion. The shaft part 103 is attached to the locking part 102 and is continuous to make a locked stable structure.

In an embodiment, the shaft part 103 can be a smooth unthreaded part or a sparse shallow threaded part or can be of standard specification for fixation. In effect the selection of threaded or unthreaded configuration of the shaft part 103 can be based on the need.

In an example embodiment, an end of the shaft 103 can optionally be thickened to effectively deliver the torque of insertion and removal and inclusion of reverse cutting threads are exercised to facilitate removal.

According to an embodiment, the shaft part 103 is either solid or cannulated and can be attached to the locking part 102 with or without hemispherical cap 101. Further, the length of the shaft is determined based on the desired site of the delivery of the agent.

In an embodiment, the shaft part 103 of the local delivery device can deliver therapeutic agent depending on the length of the shaft part 103 to an intramedullary or a far-cortex region. The shaft part 103 that is not cannulated also exerts its effects around the length of its insertion for the substances and effects of intended delivery as in piezoelectric or other mechanism on demand.

In an embodiment, the local delivery device can works in various combination such as a hemispherical cap 101 and a locking part 102 as a local delivery device for targeted therapeutic effect; or a locking part 102 and a shaft part 103 as a local delivery device for targeted therapeutic effect; or a hemispherical cap 101, a locking part 102 and a shaft part 103, all together as a local delivery device for targeted therapeutic effect.

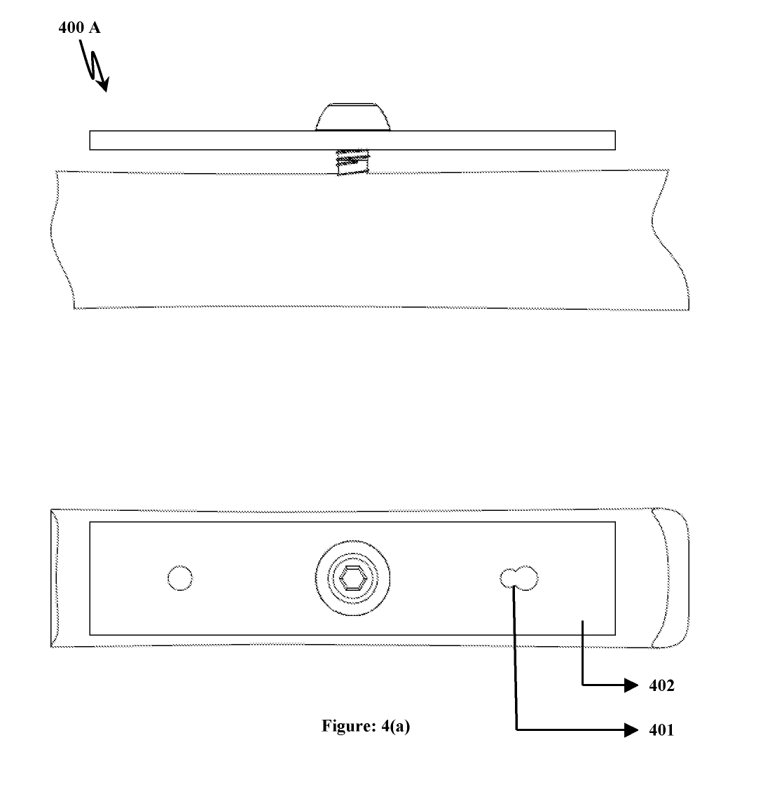

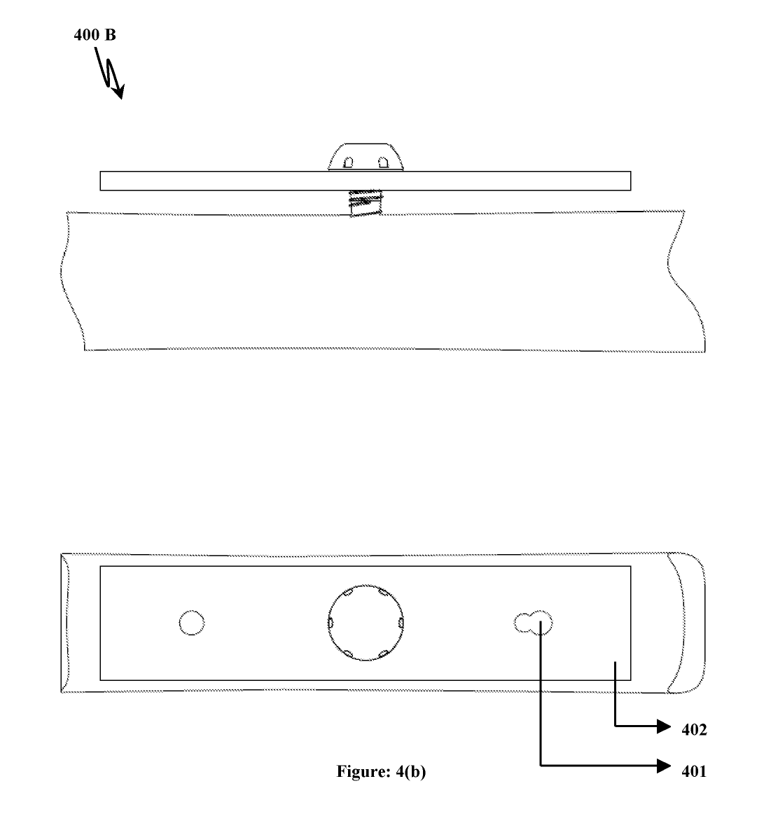

FIG. 4a illustrates a centre tightened local delivery device with a bone locking plate 400A, according to an embodiment. In an embodiment, a periphery tightened local delivery device with a bone locking plate 400B is illustrated in FIG. 4b. A threaded locking part 102 with pitch matches to the locking hole 401 of locking plate 402 for delivering therapeutic agents. The hemispherical cap 101 may also encompass and stabilize additional space expanders as in case of combi-holes, where a locking hole 401 with additional space expanders for therapeutic agent delivery stabilized by a circular hemispherical cap 101 by locking to the threaded locking part 102.

According to an embodiment, a local delivery device for targeted therapeutic effect may be a solid whole body, which are made of intended substance or their combinations and also serve as a canister to store and deliver the substances of choice. In an embodiment, the delivery device can vary in size and shape according to implant's locking dimensions and has a locking mechanism with the implant like the threads in case of locking plates or can be otherwise in accordance with the implant's locking dimensions.

In an embodiment, the local delivery device can deliver their effects to an area underneath the plate and around the plate and if bone including can deliver in addition to the above to intramedullary and far cortex region in effect almost over the entire region around its application. These make virtually every option of intended therapeutic effect and every location desired reachable to surgeon.

In an embodiment, the local delivery device can be inserted in unicortical fashion to deliver intramedullary therapeutic effect or can be inserted in biocortical fashion to have far-cortex region effect and can also have an intramedullary effect if an additional hole is given at the intramedullary region or is made of solid therapeutic agent of intended benefit.

In an embodiment, the length of the delivery device can be varied from bone sparing that is positioned exclusively to the implant without involving the bone or involving the medullar cavity and the far cortex region depending upon the need for the therapeutic delivery.

Wisdom of fracture fixation says about half of the holes of the locking plates may be left unused on either side of fracture and a communition area is spared of any fixation and also prescribe to use a plate with length more than three times the length of communition, in any case the availability of unused locking holes in the plate are plenty and spanned over an area of intended therapeutic effect delivery. The local delivery device can be used in these holes for delivering multiple agents and modalities separately or multiple agents and modalities can be delivered through a single application itself; all these can be done without altering the configuration of fixation.

According to an embodiment, the local delivery device uses the holes which are left behind after fixation of implant, the technique of insertion can be varied to the scenario that is by choosing larger drill bits than prescribed for fixation in relation to a diameter of the shaft of the local delivery device wherein fixation, stability or rigidity is not of concern but application of local delivery device at the desired location without altering the configuration of fracture reduction and fixation. The threads over the shaft 103 of local delivery device can be varied from standard specification to sparse and shallow to smooth non-threaded shaft depending on the need of the surgeon.

In an embodiment, the local delivery device is applied using a predrilled hole, which is done through a corresponding locking guide to match the locking threads to the hole.

In an embodiment, the local delivery device needs to be a part of stability in-fixation, the predrilled hole size and thread configurations can match the standard prescriptions.

In an embodiment, all the parts of the local delivery device are designed in accordance to the local need and a thickness of a wall of the device and a thread pattern are determined to establish adequate strength for insertion, continuity during an entire period of implantation and exit without fail.

In an embodiment, the local delivery device can be modified adequately for usage in various implants and prosthesis, spinal implant and intramedullary nails, suture anchors etc., with fundamentals of locking part or stabilizing part with or without augmentation of space and delivery of the substance intended benefit locally.

In an example embodiment, in case of a requirement to remove or replace the local delivery device in isolated fashion from a specific location the approach, technique and efficacy are very simple and straight forward with usage of x-ray control, zig etc., and can be done in a minimally invasive fashion using stab incisions.

In an example embodiment, the method of loading the therapeutic agent may be a pre-filled device, may also be constituted and filled intra-operatively and can also be refilled or replaced in the post implantation phase as desired.

In an example embodiment, the delivery device may be added for varying the strength of the implant either alone or with added therapeutic agents.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A device for local delivery of targeted therapeutic effect, comprising:
a cap includes a slot for tightening the device;
a locking part includes an optionally threaded outer surface to lock the device with an implant or a treatment part independently; and
a shaft part having a predetermined length extending towards desired location of in and around the treatment part.

2. The device of claim 1, wherein the locking part includes a mechanism other than threaded outer surface to lock the device with an implant or a treatment part.

3. The device of claim 1, wherein the device is made from a material selected from the group comprising of metal, alloy, polymer, bio-absorbable material, material of therapeutic agent itself with or without necessary bases, additives or adjuvant, or combination of all.

4. The device of claim 1, wherein said locking part along with cap and shaft parts either in combination or alone includes a canister to accommodate the desired agents for delivery.

5. The device of claim 1, wherein said cap, locking part and shaft part are joined as non-seperable single unit, wherein the joining is made in a smooth fashion aligned at predetermined angle preferably at obtuse angle to prevent stress raiser effect.

6. The device of claim 1, wherein said cap, locking part and shaft part are provided seperately to use or in different combination of them for the intended therapeutic effect.

7. The device of claim 1, wherein size and shape of said cap, locking part and shaft part are variable based on the application.

8. The device of claim 7, wherein shape of said cap is either spherical or polygon.

9. The device of claim 7, wherein said cap is modified to act as a scaffold for tissue regeneration and to accept S.S wire, cables, sutures as required.

10. The device of claim 7, wherein said shaft part includes either threaded outer surface or smooth unthreaded outer surface to suite the need of treatment part.

11. The device of claim 7, wherein said shaft part includes reverse cutting threads to facilitate the easy removal of the device.

12. The device of claim 7, wherein said cap, locking part and shaft part are provided in solid base to function as a reservior or vehicle and made of the substances intended.

13. The device of claim 7, wherein said cap, locking part and shaft part optionaly include one or more apertures to facilitate release of substance.

14. The device of claim 13, wherein said release of substance from the apertures is regulated by physical structures includes of sieves, membranes or plugs, or by physical and chemical properties of the materials includes of viscosity, charged ions, pH and biological interaction with body fluids.

15. The device of calim 13, wherein the substance is prefilled in the device or constituted intra-operatively and applied or can replenish or replace the one existing inside the device.

16. The device of claim 1, wherein said slot for driver is provided in center of the cap, wherein the said slot optionally augumented at its base extending in middle up to the farthest part of the device to deliver the torque effectively.

17. The device of claim 1, wherein said slot for driver is provided in periphery of the cap, wherein said slot optionally augumented at its periphery to the fartherst possible to deliver the torque effectively.

18. The device of claim 16 or claim 17, wherein a torque controlled driver used for fastening the device using said slot to prevent complications in the removal of implantation.

19. The device of claim 1, wherein said device is present in a combination or together applied along with a bone locking plate, or other implants of choice or parts of the delivery device are used in combination or together as an individual delivery device at the desired part.

20. The device of claim 1, wherein said device is used to deliver physical, pharmacological, biological active substances, piezo electric materials, ion diffusion, galvanic particulates, nano particles or things to aid in fracture healing, clearing infection or malignancy and to strengthen bone and soft tissues, cartilage regeneration, oseoconductive, osteoinductive materials, bone graft and bone graft substitutes.
